(19) United States
(12) Patent Application Publication
(10) Pub. No.: US 2007/0293784 A1
(43) Pub. Date: Dec. 20, 2007

(54) TEMPERATURE MONITORING APPARATUS

(75) Inventor: Chien-Sheng Chiu, Taichung (TW)

Correspondence Address: DARBY & DARBY P.C., P.O. BOX 770, Church Street Station, New York, NY, 10008-0770, US

(73) Assignee: SYRIS TECHNOLOGY CORP., Taichung (TW)

(21) Appl. No.: 11/424,341

(22) Filed: Jun. 15, 2006

(51) Int. Cl. A61B 5/00

(52) U.S. Cl. 600/549

(57) ABSTRACT

A temperature monitoring apparatus includes a casing, a detector, a sensor, a controller unit, and an indicator unit. The casing is formed with a hole therethrough. The detector is disposed in the casing, and detects extension of an object into the hole in the casing. The sensor is disposed in the casing, and is adapted to measure a temperature of the object extended into the hole in the casing. The controller unit is coupled to the detector and the sensor, and is operable so as to generate an actual temperature value with reference to the temperature measured by the sensor. The indicator unit is coupled to and controlled by the controller unit so as to provide an indication when the detector detects absence of the object in the hole in the casing.

### BACKGROUND OF THE INVENTION

1. Field of the Invention

The invention relates to a temperature monitoring apparatus, more particularly to a temperature monitoring apparatus that is capable of making continuous measurements of a body temperature of a patient.

2. Description of the Related Art

Infrared ear thermometers are widely used for measuring a body temperature of a patient. During use, a short tube, with a protective sleeve, is inserted into the ear of the patient, and a button is pressed to actuate an infrared detector. Thereafter, the infrared ear thermometer beeps, and a readout of the body temperature of the patient is produced on a liquid crystal display.

Although the known infrared ear thermometer achieves its intended purpose, since the body temperature of the patient has to be routinely monitored, the use of the known infrared ear thermometer causes inconvenience on the part of a caregiver. To solve this problem, it has been proposed to use a thermometer that is attached securely to the patient. This, however, can cause other problems. Particularly, when the proposed thermometer is not properly attached to the patient, an inaccurate result may be obtained.

### SUMMARY OF THE INVENTION

Therefore, the object of the present invention is to provide a temperature monitoring apparatus that can overcome the aforesaid drawbacks of the prior art.

According to the present invention, a temperature monitoring apparatus comprises a casing, a detector, a sensor, a controller unit, and an indicator unit. The casing is formed with a hole therethrough. The detector is disposed in the casing, and is adapted to detect extension of an object into the hole in the casing. The sensor is disposed in the casing, and is adapted to measure a temperature of the object extended into the hole in the casing. The controller unit is coupled to the detector and the sensor, and is operable so as to generate an actual temperature value with reference to the temperature measured by the sensor. The indicator unit is coupled to and controlled by the controller unit so as to provide a first indication when the detector detects absence of the object in the hole in the casing, and a second indication when the actual temperature value generated by the controller unit is within a predetermined temperature range.

### BRIEF DESCRIPTION OF THE DRAWINGS

Other features and advantages of the present invention will become apparent in the following detailed description of the preferred embodiment with reference to the accompanying drawings, of which:

FIG. 1 is an exploded perspective view of the preferred embodiment of a temperature monitoring apparatus according to the present invention;

FIG. 2 is a schematic circuit block diagram of the preferred embodiment;

FIG. 3 is a sectional view of the preferred embodiment in an assembled state; and

FIG. 4 is a schematic view of the preferred embodiment to illustrate a sensor that measures ambient temperature through a hole in a casing.

### DETAILED DESCRIPTION OF THE PREFERRED EMBODIMENT

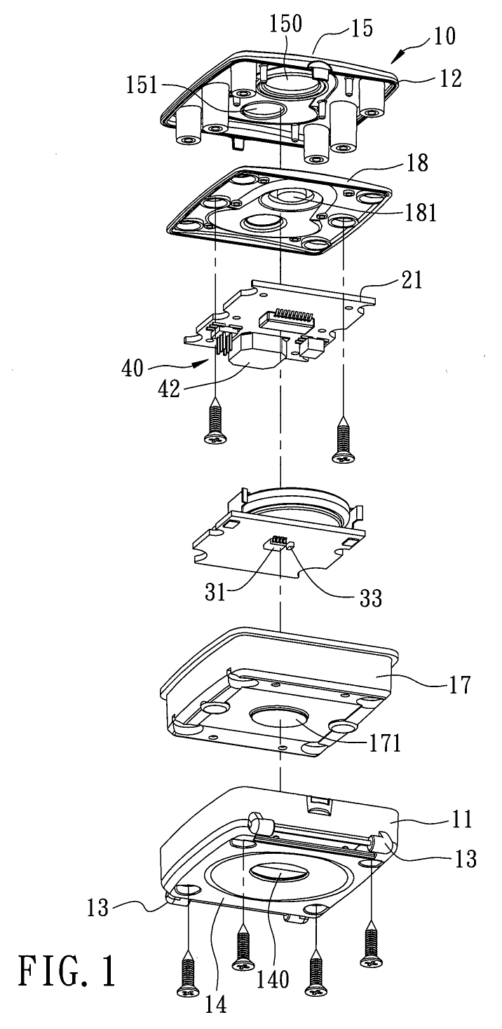

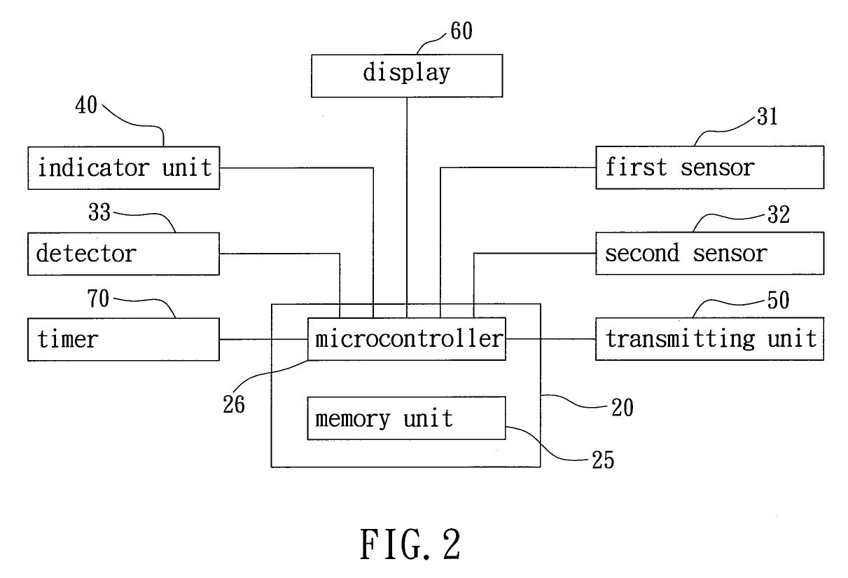

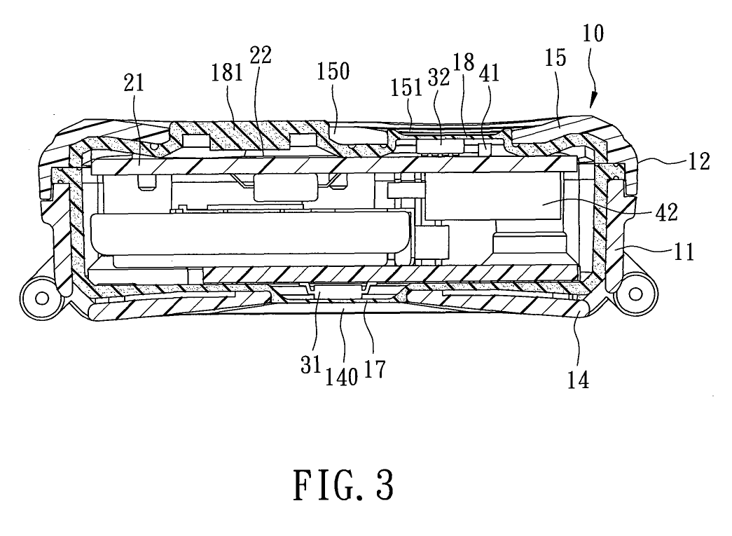

Referring to FIGS. 1 to 3, the preferred embodiment of a temperature monitoring apparatus according to this invention is shown to include a casing 10, a detector 33, first and second sensors 31, 32, a controller unit 20, and an indicator unit 40.

The temperature monitoring apparatus of this embodiment is applicable for monitoring a body temperature of a patient (not shown), in a manner that will be described hereinafter.

The casing 10 includes a first base wall 14 that is formed with a first hole 140 therethrough, a second base wall 15 that is opposite to the first base wall 14 and that is formed with a second hole 150 therethrough, a first surrounding wall 11 that extends from and that surrounds the first base wall 14, and a second surrounding wall 12 that extends from and that surrounds the second base wall 15.

The detector 33 is disposed in the casing 10, and detects extension of a skin of the patient into the first hole 140 in the first base wall 14 of the casing 10. In this embodiment, the detector 33 is a light detector that detects ambient light condition in the casing 10. Thus, when the detector 33 detects a dark ambient light condition, it concludes that the skin of the patient is extended into the first hole 140 in the first base wall 14 of the casing 10. On the other hand, when the detector 33 detects a bright ambient light condition, it concludes the absence of the skin of the patient in the first hole 140 in the first base wall 14 of the casing 10.

In an alternative embodiment, the detector 33 is an electrostatic detector that detects static electricity produced by the skin of the patient. That is, when the detector 33 detects the static electricity, it concludes that the skin of the patient is extended into the first hole 140 in the first base wall 14 17 of the casing 10. On the other hand, when the detector 33 does not detect any static electricity, it concludes the absence of the skin of the patient in the first hole 140 in the first base wall 14 of the casing 10.

The casing 10 further includes a pair of strap loop connectors 13, each of which is provided on a respective one of opposite sides of the first surrounding wall 11 of the casing 10, for connecting with a strap (not shown) that is tied around the wrist of the patient. As such, the temperature monitoring apparatus of this invention can be attached securely to the patient to thereby squeeze the skin of the patient into the first hole 140 in the first base wall 14 of the casing 10.

The first sensor 31 is disposed in the casing 10, and measures a temperature of the skin of the patient extended into the first hole 140 in the first base wall 14 of the casing 10.

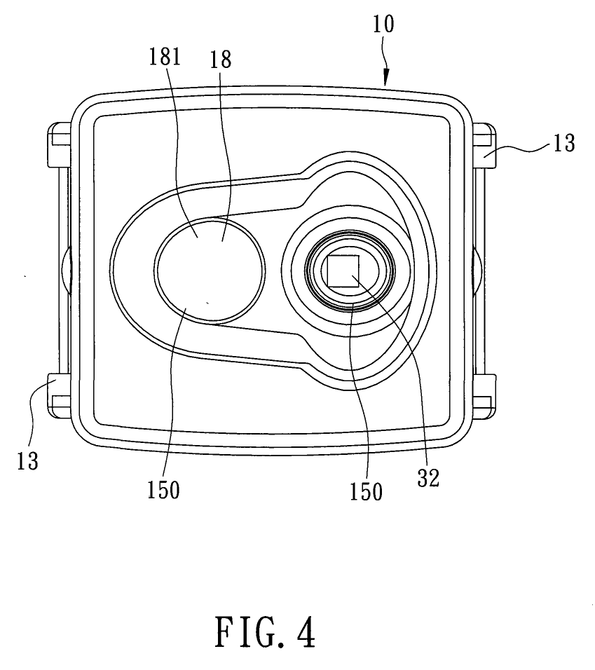

The second sensor 32 is disposed in the casing 10, and measures an ambient temperature through the second hole 150 in the casing 10, as best shown in FIG. 4.

In this embodiment, each of the first and second sensors 31, 32 is a temperature sensor, model no. ADT7301, implemented in an integrated circuit.

The controller unit 20 includes a microcontroller 26 and a memory unit 25. The microcontroller 26 of the controller unit 20 is connected electrically to the detector 33 and the first and second sensors 31, 32. In this embodiment, the microcontroller 26 of the controller unit 20 is under the model no. MSP430F2131. The memory unit 25 of the controller unit 20 is connected electrically to the microcontroller 26 of the controller unit 20 for storing object data and temperature correcting factors therein. In this embodiment, the memory unit 25 includes a SPI serial EEPROMS under the model no. AT25320.

It is noted that the object data is pertinent to the patient whose temperature is to be monitored, such as name, age, telephone number, address, etc. Furthermore, each temperature correcting factor corresponds to an age group of a specific patient. That is, for patients in the age group of 1 to 19 years, the temperature correcting factor is −0.2° C. For patients in the age group of 20 to 50 years, the temperature correcting factor is 0° C. For patients in the age group of above 50 years, the temperature correcting factor is +0.2° C.

The microcontroller 26 of the controller unit 20 is operable so as to generate an actual temperature value with reference to the temperature measured by the first sensor 31, the ambient temperature measured by the second sensor 32, and the temperature correcting factors in the memory unit 25 of the controller unit 20. In particular, the microcontroller 26 of the controller unit 20 first compares the temperature measured by the first sensor 31 with the ambient temperature measured by the second sensor 32, and generates a comparison result. Then, the microcontroller 26 of the controller unit 20 adds the temperature correcting factor, which corresponds to the age group of the patient, to the comparison result to result in the actual temperature value. As such, the actual temperature value generated by the microcontroller 26 of the controller unit 20 is an accurate indication of a body temperature of the patient.

The indicator unit 40 is coupled to and controlled by the microcontroller 26 of the controller unit 20 to provide a first indication when the detector 33 detects the absence of the skin of the patient in the first hole 140 in the first base wall 14 in the casing 10, and a second indication when the actual temperature value generated by the microcontroller 26 of the controller unit 20 is within a predetermined temperature range. In this embodiment, the indicator unit 40 includes a light-emitting device 41, preferably a light-emitting diode, that provides the first indication by emitting a continuous light and the second indication by generating a flashing light, and a sound-generating device 42, preferably a buzzer, that provides the first indication by generating a continuous sound and the second indication by generating an intermittent sound.

It is noted that when the temperature monitoring apparatus of this invention is properly attached to the patient, the skin of the patient extends into the first hole 140 in the first base wall 14 of the casing 10. As such, it can be ensured that the first temperature measured by the first sensor 31 indeed belongs to the patient. However, when the temperature monitoring apparatus is accidentally detached from the patient, the detector 33 detects the absence of the skin of the patient in the first hole 140 in the first base wall 14 in the casing 10, which causes the indicator unit 40 to provide the first indication. This, in turn, alerts the patient (or a caregiver) that the temperature monitoring apparatus needs to be properly reattached to the patient.

In addition, in this embodiment, the predetermined temperature range is above a normal temperature range of a person. As such, when the actual temperature value generated by the microcontroller 26 of the controller unit 20 is within the predetermined temperature range, it suggests that the patient has a fever.

In an alternative embodiment, the indicator unit 40 includes a vibration-producing device (not shown), such as a vibrating motor, that is coupled to and that is controlled by the microcontroller 26 of the controller unit 20 to provide the first indication by producing a continuous vibration and the second indication by producing an intermittent vibration.

The temperature monitoring apparatus further includes a first thin film 17 that is disposed in the casing 10, and that is formed with a projection 171 extending into the first hole 140 in the first base wall 14 of the casing 10. The construction as such prevents water and dust from entering the casing 10.

The controller unit 20 further includes a circuit 21, and a switch 22 that is actuatable to control operation of the circuit 21. In particular, the second base wall 15 of the casing 10 is further formed with an access hole 151 therethrough. The temperature monitoring apparatus further includes a second thin film 18 that is disposed in the casing 10, that is formed with a projection 181 extending into the access hole 151 in the second base wall 15 of the casing 10, that is accessible from the outside of the casing 10, as best shown in FIG. 4, that is provided with conductive coating (not shown), and that is deformable so as to permit actuation of the switch 22.

It is noted that the first and second thin films 17, 18 are so thin and are almost transparent so as not to affect adversely operations of the detector 33 and the first and second sensors 31, 32.

The temperature monitoring apparatus further includes a transmitting unit 50 that is connected electrically to and that is controlled by the microcontroller 26 of the controller unit 20 to wirelessly transmit the actual temperature value generated by the microcontroller 26 of the controller unit 20 to a server (not shown). As such, status of the patient can be monitored from afar. In this embodiment, the transmitting unit 50 includes a radio frequency integrated circuit (RF IC) under the model no. CC2500.

The temperature monitoring apparatus further includes a display 60 that is coupled to and that is controlled by the microcontroller 26 of the controller unit 20 to show the actual temperature value generated by the microcontroller 26 of the controller unit 20. In this embodiment, the display 60 is a liquid crystal display (LCD).

The temperature monitoring apparatus further includes a timer 70 that is coupled to and that is controlled by the microcontroller 26 of the controller unit 20 to generate the current time of day. The display 20 is further controlled by the microcontroller 26 of the controller unit 20 to show the current time of day generated by the timer 70. In this embodiment, the timer 70 is software that is executed by the microcontroller 26 of the controller unit 20.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A temperature monitoring apparatus, comprising:
a casing formed with a first hole therethrough;
a detector disposed in said casing, and adapted to detect extension of an object into said first hole in said casing;
a first sensor disposed in said casing, and adapted to measure a temperature of the object extended into said first hole in said casing;
a controller unit coupled to said detector and said first sensor, and operable so as to generate an actual temperature value with reference to the temperature measured by said first sensor; and
an indicator unit coupled to and controlled by said controller unit so as to provide a first indication when said detector detects absence of the object in said first hole in said casing, and a second indication when the actual temperature value generated by said controller unit is within a predetermined temperature range.

2. The temperature monitoring apparatus as claimed in claim 1, wherein said casing is further formed with a second hole, said temperature monitoring apparatus further comprising a second sensor that is coupled to said controller unit and that is adapted to measure an ambient temperature through said second hole in said casing, said controller unit generating the actual temperature value with reference to the temperature measured by said first sensor and the ambient temperature measured by said second sensor.

3. The temperature monitoring apparatus as claimed in claim 2, wherein said casing includes opposite first and second base walls, and a surrounding wall that extends from and that surrounds one of said first and second base walls, each of said first and second holes being formed in a respective one of said first and second base walls.

4. The temperature monitoring apparatus as claimed in claim 3, wherein said casing further includes a pair of strap loop connectors, each of which is provided on a respective one of opposite sides of said casing, for connecting with a strap that is tied around the object.

5. The temperature monitoring apparatus as claimed in claim 1, further comprising a thin film disposed in said casing, and formed with a projection that extends into said first hole in said casing to thereby prevent water and dust from entering said casing.

6. The temperature monitoring apparatus as claimed in claim 1, wherein said casing is further formed with an access hole therethrough, said controller unit including a circuit, and a switch that is actuatable to control operation of said circuit, said temperature monitoring apparatus further comprising a thin film disposed in said casing, and formed with a projection that extends into said access hole in said casing and that is deformable so as to permit actuation of said switch.

7. The temperature monitoring apparatus as claimed in claim 1, further comprising a memory unit coupled to said controller unit for storing object data pertinent to the object whose temperature is to be monitored.

8. The temperature monitoring apparatus as claimed in claim 7, wherein said memory unit further stores a temperature correcting factor therein, said controller unit generating the actual temperature value with reference to the temperature measured by said first sensor and the temperature correcting factor in said memory unit.

9. The temperature monitoring apparatus as claimed in claim 1, wherein said first sensor is a temperature sensor implemented in an integrated circuit.

10. The temperature monitoring apparatus as claimed in claim 2, wherein said second sensor is a temperature sensor implemented in an integrated circuit.

11. The temperature monitoring apparatus as claimed in claim 1, wherein said detector is one of a light detector and an electrostatic detector.

12. The temperature monitoring apparatus as claimed in claim 1, wherein said indicator unit includes at least one of a light-emitting device, a sound-generating device, and a vibration-producing device.

13. The temperature monitoring apparatus as claimed in claim 1, further comprising a transmitting unit coupled to and controlled by said controller unit to wirelessly transmit the actual temperature value generated by said controller unit.

14. The temperature monitoring apparatus as claimed in claim 1, further comprising a display coupled to and controlled by said controller unit to show the actual temperature value generated by said controller unit.

15. The temperature monitoring apparatus as claimed in claim 14, further comprising a timer coupled to and controlled by said controller unit to generate the current time of day, said display being further controlled by said controller unit to show the current time of day generated by said timer.
